(19) United States
(12) Patent Application Publication
(10) Pub. No.: US 2005/0228382 A1
(43) Pub. Date: Oct. 13, 2005

(54) Screw and rod fixation assembly and device

(76) Inventors: Marc Richelsoph, Bartlett, TN (US); Joseph Clift, Bartlett, TN (US)

Correspondence Address: Amy E. Rinaldo;KOHN & ASSOCIATES, PLLC, Suite 410, 30500 Northwestern Highway, Farmington Hills, MI, 48334, US

(21) Appl. No.: 10/822,571

(22) Filed: Apr. 12, 2004

(51) Int. Cl. A61B 17/58

(52) U.S. Cl. 606/61

(57) ABSTRACT

A screw and rod fixation assembly for fixing a screw and a rod, the assembly having a screw including a screw head, a body portion including a screw head receiving device for receiving the screw head and a rod receiving device for receiving a rod therein, the rod receiving device being offset from the screw head receiving device, and a locking device for locking a rod within the rod receiving device. A medical device for fixing a screw and a rod, the assembly having a screw including a screw head, a body portion having a receiver for receiving the screw head and an offset slot for receiving a rod therein, and a lock for locking the rod within the assembly. A receiving member for receiving and maintaining a rod therein, the receiving member having a body portion having a receiver for receiving a screw head and an offset slot for receiving a rod therein. A rod receiving device having a body portion having a receiver for receiving a screw therein and a rod seat offset within the body portion.

### BACKGROUND OF THE INVENTION

1. Technical Field

The present invention relates to orthopedic devices. More particularly, the present invention relates to screw and rod fixation assemblies for use with bone fixation systems.

2. Background Art

Several techniques and systems have been developed for correcting and stabilizing the spine and for facilitating fusion at various levels of the spine. Stabilization of the spine for various conditions, including degenerative disk disease, scoliosis, spondylolisthesis, and spinal stenosis, often require attaching implants to the spine and then securing the implants to spinal rods. Such spinal fixation devices can immobilize the vertebrae of the spine and can alter the alignment of the spine over a large number of vertebrae by connecting at least one elongate rod to a sequence of selected vertebrae. The rods can span a large number of vertebrae. The spine anatomy, however, rarely allows for three or more implants to be directly in line. In order to allow for this irregularity, the rod must be contoured.

Spinal fixation has become a common approach in the fusion of vertebrae and treating fractures and the above listed spinal disorders. A common device used for spinal fixation is a bone fixation plate assembly. Typical bone fixation plate assemblies have a relatively flat, rectangular plate with a plurality of apertures therethrough. Other devices include an implantation fixation system that locks a rod to several vertebrae. In the assemblies, as in with other spinal fixation systems, various fasteners, such as bone screws, are used to secure the bone fixation plate assembly or the implantation fixation assembly to the desired and targeted vertebrae of the patient. The screws vary in design and shape depending upon their desired location and use thereof.

Preferably, polyaxial locking screws are used to secure the device to vertebral boney tissue. The key to the polyaxial screws used with the above systems is having a screw capable of being securely fastened to the vertebrae and to the assembly thereof. The polyaxial screws must be used in conjunction with a type of screw head securing device that provides a strong lock to the polyaxial screw. Any movement of the screw can be detrimental towards the healing process of the spine. Further, additional damage can occur if there is movement of the screw once it has been fixed to the vertebrae. Therefore, movement of the screw must be minimized or eliminated.

There are numerous polyaxial screws existing in the market today and known in the prior art. Additionally, numerous devices exist that provide a securing means for locking the polyaxial screw. For example, U.S. Pat. No. 5,554,157, U.S. Pat. No. 5,549,608, and U.S. Pat. No. 5,586,984 all to Errico et al. disclose polyaxial locking screws and coupling element devices for use with a rod fixation apparatus. The '157 patent discloses a coupling element including an interior axial passage having an interior surface that is inwardly curvate at the lower portion thereof such that it comprises a socket for polyaxially retaining a spherical head of a screw. The coupling element further includes a pair of vertically oriented opposing channels extending down from the top of the coupling element that define a rod seat therebetween. The channel further creates walls leading to a pair of upwardly extending members, each including an exterior threading disposed on the uppermost portion thereof for receiving a locking nut. During the implantation of the assembly, the locking nut seals against the top of the rod that, in turn, is seated on top of the screw head. The nut causes the rod to be locked between the nut and the screw and the screw to be locked in the socket.

The '608 patent discloses a modification wherein a locking ring is disposed about the exterior of the lower portion of the coupling element and provides an inward force on an outwardly tapered portion upon downward translation thereof. As a result, the interior chamber crush locks a screw head therein to eliminate the polyaxial nature of the screw element coupling.

The '984 patent discloses a polyaxial orthopedic device including a coupling element having a tapered lower portion having a slotted interior chamber in which a curvate head of a screw is initially polyaxially disposed. The coupling element includes a recess for receiving a rod of the implant apparatus. A locking ring is disposed about the lower portion of the coupling element and provides an inward force on the outwardly tapered portion upon downward translation thereof. The vertical slots are caused to close and crush, thereby locking the screw head within the interior chamber thereof.

U.S. Pat. No. 6,280,442 to Barker et al. discloses a complex locking mechanism having a screw head with complex head geometry, a crown member, and an outer rigid body. Locking occurs by compressing the crown member against the complex head, which compresses the head against the rigid seat. The compression crushes the machine ridges on the head and secures the screw therein.

Another example of a common locking mechanism is a type of collet that includes a spherical seat with a flexible portion that is designed to deflect around the screw. By compressing the flexible portion against a rigid, outer wall, the collet is compressed against the head to cause locking therein. Examples of collets are found in numerous patents. For example, U.S. Pat. No. 6,053,917 to Sherman et al. discloses a multiaxial bone screw assembly that includes a bone screw having a partially spherical head. Additionally, the assembly includes a receiver member that has a central bore that defines a tapered recess to receive a contracting collet carrying the head of the bone screw. The collet defines a partially spherical recess to receive the head of the bone screw and includes deflectable fingers that substantially surround the screw head. As a set screw is tightened into the receiver member, the set screw compresses the rod against the collet, which presses the collet into the tapered recess of the receiver member, thereby deflecting the fingers of the collet against the bone screw head.

Another patent, U.S. Pat. No. 5,964,760 to Richelsoph, discloses a spinal implant fixation assembly that includes a bone fixation member. A rod seat is operatively connected to the bone fixation element for seating a portion of a rod therein. A locking mechanism, in the form of a nut and locking ring, engages the rod seat for forcing an inner wall of the rod seat to contour around and engage the rod seated therein and for locking and fixing the rod relative to the inner housing. The assembly further includes a screw head receiving insert for retaining a head of a screw therein. The insert is moveable within the assembly between a locked position entrapping the screw head and an unlocked position wherein the screw head enters or escapes.

Other polyaxial screw devices that include similar collets are disclosed in U.S. Pat. No. 6,010,503 to Richelsoph, U.S. Pat. No. 5,910,142 to Tatar, which discloses the use of a spherical collet that is compressed between the screw head and the rod, and U.S. Pat. No. 5,891,145 to Morrison et al., which discloses the use of a very complex double wedge locking mechanism.

More specifically, the '142 patent to Tatar discloses a polyaxial pedicle screw device for use with a rod implant apparatus, which utilizes a rod mounted ferrule. The device further includes a screw having a curvate head and a rod receiving body. The body has a rod receiving channel and an axial bore into which the head of the screw is inserted. The rod mounted ferrule seats into a small curvate recess in the upper portion of the screw head such that the rod may enter the body at a variety of angles while maintaining secure seating against the head of the screw. The insertion of a top, set-screw compresses down on the ferrule, locking the rod in position and onto the screw head. Further, the body is locked in position to completely secure the assembly.

The '145 patent to Morrison et al. discloses a spinal fixation assembly that includes a bone engaging fastener and an elongated member such as a spinal rod. The fixation assembly is a multiaxial assembly that permits fixation of the bone engaging fastener to the spinal rod at any of the continuous ranges of angles relative to the rod in three dimensional space. The fixation assembly includes a receiver member having a bore therethrough; the walls of which are tapered near the bottom, and a channel communicating with the bore and having an upper opening at the top of the receiver member for insertion of a spinal rod. An outer wedge member and an inner wedge member are also included. Both members have the general shape of a washer and a bore therethrough. In each wedge member, the respective bore is not parallel to the central axis of the respective wedge member. Additionally, the outside surfaces of the wedge members may be tapered and the respective bores may be tapered so as to self-lock when seated and tightened. The bone engaging fastener fits within the bore of the inner wedge member, which in turn fits within the bore of the outer wedge member, which in turn fits within the tapered sides of the receiver member. When the desired position of the bone engaging fastener in three dimensional space is attained, the components are seated to achieve a tight friction fit.

U.S. Pat. No. 6,063,089 to Errico et al. discloses a polyaxial orthopedic device for use with a rod implant apparatus that includes a screw having a head, a tubular body having holes on top, side, and bottom thereof, and a rod coupling element. The head of the screw is disposed in the body with the shaft of the screw extending out of the bottom hole, such that the body and the screw may initially rotate relative to one another. The rod coupling element has a ball shaped end that sits in the body with the remainder of the rod coupling element extending out of the side hole of the body, such that the rod coupling element and the body are initially polyaxially coupled relative to one another. The ball end of the rod coupling element is disposed on top of the head of the screw. A set screw is provided on top of the body, the tightening of which causes the ball, head, and body to be crush locked together, thereby preventing further relative motion.

In the prior art, particularly those described herein, the polyaxial screw assemblies include a complex locking mechanism and additional locking parts to prevent the movement of the polyaxial screw. Typically, the more complex the locking mechanism requires larger components and manufacturing costs are expensive. Locking and achieving strong hold values become more difficult with more parts. Further, the sizes of the various fixation plates and fixation assemblies are critical to these types of surgeries. Bulky components can cause soft tissue irritation, as well as compromise the facet joints at the end of a fusion. Minimizing the size of the implants used is critical in spinal surgery. Soft tissue irritation resulting from extensions of implants is a common occurrence. Many times, it is caused by the implant being large relative to its environment. For example, implants can be too large to be sufficiently covered within the muscle tissue. Hence, a reduction in the overall dimensions of the implant is a critical advantage.

Accordingly, there is a need for a screw head securing mechanism or device that provides a strong, effective, and secure lock of the screw head in its desired position. Additionally, there is a need for a screw head securing mechanism or device that is minimal in size and has a reduced amount of components to provide for a simpler, more effective, and less cumbersome device for fixing screws.

### SUMMARY OF THE INVENTION

According to the present invention, there is provided a screw and rod fixation assembly for fixing a screw and a rod, the assembly having a screw including a screw head, a body portion including a screw head receiving device for receiving the screw head and a rod receiving device for receiving a rod therein, the rod receiving device being offset from the screw head receiving device, and a locking device for locking a rod within the rod receiving device. Also provided is a medical device for fixing a screw and a rod, the assembly having a screw including a screw head, a body portion having a receiver for receiving the screw head and an offset slot for receiving a rod therein, and a lock for locking the rod within the assembly. A receiving member for receiving and maintaining a rod therein, the receiving member having a body portion having a receiver for receiving a screw head and an offset slot for receiving a rod therein is provided. A rod receiving device having a body portion having a receiver for receiving a screw therein and a rod seat offset within the body portion is also provided.

### DESCRIPTION OF THE DRAWINGS

Other advantages of the present invention are readily appreciated as the same becomes better understood by reference to the following detailed description when considered in connection with the accompanying drawings wherein:

FIG. 1 is a side view of the device of the present invention in a non-engaged position;

FIG. 2 is a side view of the device of the present invention in an engaged position;

FIG. 3 is a side view of the device of the present invention including a cam lock assembly;

FIG. 4 is a side view of the device of the present invention in an open position;

FIG. 5 is a side view of the device of the present invention in a locked position;

FIG. 6 is a detail view of the cam lock shown in FIG. 3;

FIG. 7 is a side view of the device of the present invention in an open position with a rod in place;

FIG. 8 is a cut-away view of the device of the present invention FIG. 9 is a side view of the screw seat of the present invention with a wedge-shape locking surface;

FIG. 10 is a side view of the device of the present invention including the wedge-shaped screw seat without the inclusion of a rod;

FIG. 11 is a partial side view of the device of the present invention in a locked position;

FIG. 12 is a partial side view of the device of the present invention including the wedge-shaped screw seat having a partial body lip and without the inclusion of a rod;

FIG. 13 is a partial side view of the device of the present invention including the wedge-shaped screw seat having a full body lip and without the inclusion of a rod;

FIG. 14 is a partial side view of the device of the present invention including the wedge-shaped screw seat having a full body lip with an extraction feature and without the inclusion of a rod;

FIG. 15 is a detail view of the cam lock including a locking tab;

FIG. 16 is a side view of an alternative embodiment of the device of the present invention having threads, the device being in an unlocked position;

FIG. 17 is a side view of an alternative embodiment of the device of the present invention having threads, the device being in a locked position;

FIG. 18 is a front view of the cam lock of the present invention including a helical groove;

FIG. 19 is a side view of the cam lock of the present invention including a helical groove;

FIG. 20 is a front view of the cam lock of the present invention including a helical groove and a slot; and

FIG. 21 is a side view of an alternative embodiment of the assembly of the present invention including a helical groove.

### DETAILED DESCRIPTION OF THE INVENTION

A screw and rod fixation assembly constructed in accordance with the present invention is generally indicated as 10 in the Figures. Generally, the screw and rod fixation assembly 10 fixes and/or locks together a screw 12 and a rod 16.

The term “elastic range” as used herein is the limit of stress within which deformation of a body of material completely disappears after the removal of stress, tension, and/or force upon the body of material. Elasticity is the ability of a material to return to its original dimensions after removal of stress, tension, or force placed upon the material. Once the stress, tension, or forces exceed the elastic range however, the material cannot return to its original dimensions and is forever deformed.

The term “deformation range” as used herein means the excessive amount of strain, force, or load outside the elastic range wherein deformation is maintained and the body of material no longer can return to its original dimensions. Basically, deformation occurs wherein a change in form is produced by external forces or loads that act on the body of material.

The term “screw” 12 as used herein indicates a simple machine of the inclined plane type including a spirally grooved solid cylinder and a correspondingly grooved hollow cylinder into which it fits. The screw 12 can be any type of screw such as a set screw and can be any size, shape, or design as is known to those of skill in the art. In certain cases, it can be desirable for the screw 12 to include a screw head 22 of another shape and/or size.

The term “rod” 16 as used herein means a substantially cylindrical body having a desired length. With regard to the present invention, the rod 16 is typically a rod 16 capable of being used within a human body to aid in the alignment of bones, particularly the vertebrae.

One important feature of the screw fixation assembly 10, and more particularly a body portion 18 of the assembly 10, is the use of a semi-flexible material as opposed to a rigid material. As is known to those of skill in the material strength art, materials such as metals and composites thereof have an elastic range and a deformation range. For example, if a sample of a material is placed in tension and not allowed to exceed the elastic range, the sample will stretch.

Elastic stretching occurs in such a manner that upon release of the tension, the material will return to its original dimensions. By adapting this property to the present invention, the wall 19 of the body portion 18 acts as a controllable spring that can effectively be used to exert a uniform compressive load on a head portion of a screw, shaft, or other component. The elastic stretching occurs by introducing a screw head 22 that is larger than the internal dimensions 20 of the body portion 18. This forces the body portion 18 to expand to accept the screw head 22. In a similar fashion as a spring, the body portion 18 would return to its original dimensions upon removal of the screw head 22. However, while the screw head 22 is within the body portion 18, the body portion 18 exerts a compressive force on the screw head 22. Thus, the body portion 18 operates by inherently gripping the screw head 22 rigidly, preventing movement of the screw head 22 without the application of force or manipulation of the body portion 18 by another means.

The property of the expansion of the wall 19 of the body portion 18 within its elastic range and the resultant force generated inward around the screw head 22 is referred to as a “spring reaction.” Outside of this elastic range, the material permanently deforms and will not return to its original dimensions. Therefore, it is essential to stay within the elastic range of the material in order to have a controllable reaction in which to use the compressive forces created therein.

The compressive forces created by the expansion of the wall 19 of the body portion 18 can be readily controlled by altering the material, material thickness, coatings, or otherwise modifying the material of the body portion 18. The screw head 22 can also be altered to be of any material and hardness. A softer material allows for more compensation for tolerances on the dimensions of the components, while a harder material provides a more controlled expansion of the body portion 18.

The present invention can be constructed from any suitable material known to those of skill in the art. Preferably, the present invention is constructed from material that is compatible with use in the environments into which they are placed. The present invention can be constructed of various metallic materials that include, but are not limited to, titanium, stainless steel, and any other suitable metallic alloys known to those of skill in the art. Additional materials can also be utilized either alone or in combination with the above-described metallic materials. For instance, various plastics, fibers, and other polymers can be used in conjunction with the various metallic materials. The combinations can be used to construct various components or portions of the present invention. Moreover, coatings can be placed on various portions of the present invention in order to improve durability, strength, and utilization thereof. Typically though, any of the material used to construct the present invention should be very strong, non-reactive, and non-antigenic to biological systems if they are used in that environment. If the present invention is used outside of biological systems however, the aforementioned characteristics are not necessarily required.

There are numerous embodiments of the present invention. As such, similar structures amongst the several embodiments are shown by prime numbers in the various Figures. Although there are numerous embodiments disclosed and described herein, each of the embodiments includes a variation of the body portion, rod seat, and locking mechanism of the present invention.

Generally, the assembly 10 of the present invention includes a screw 12, a locking mechanism 14, a rod 16, and a body portion 18. The screw 12 includes a screw head 22 and a screw gripping device 26. Preferably, the screw gripping device is threaded portion 30.

The locking mechanism 14 is a lock body 54 that includes a head 66 and a base 67. The head 66 includes a relieved wall 57 and a grooved wall 56. The grooved wall 56 is capable of engaging the rod 16 and can be shaped as a wedge 58. The head 66 also includes a lock mechanism removal device 70 and a locking tab 68 for locking the locking mechanism 14 within the body portion 18. Additionally, the head 66 includes an arm 66 for maintaining the rod 16 in place. The base 67 includes a screw head seat 55. The seat 55 can be formed in any shape known to those of skill in the art to be capable of engaging the top of the screw head 22. For example, the seat 55 can be conical or spherical. The seat 55 enables the screw head 22 to move within a limited range without backing out of the body portion 18.

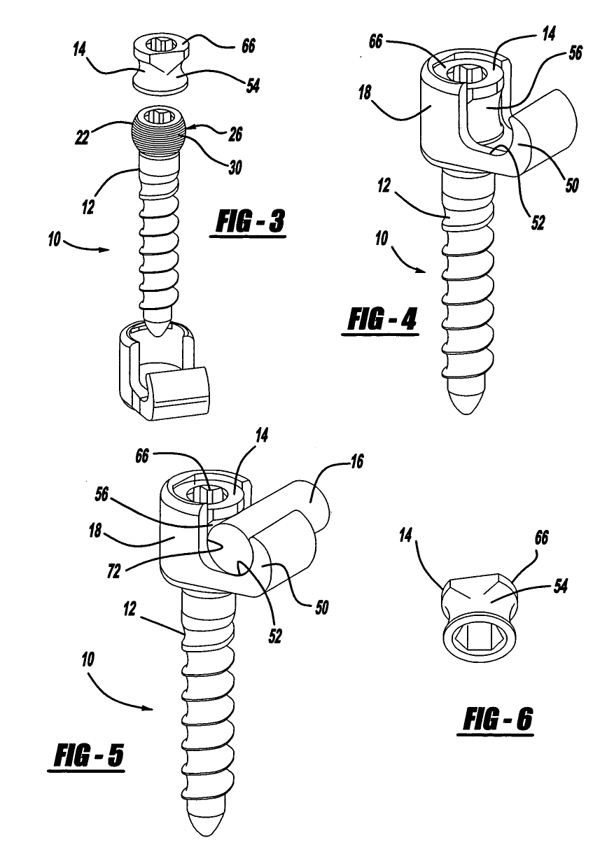

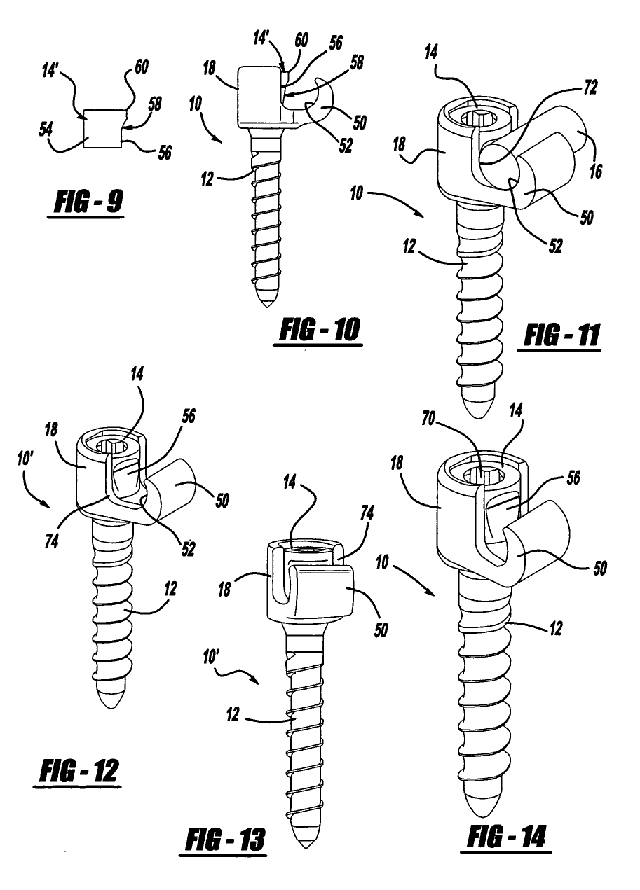

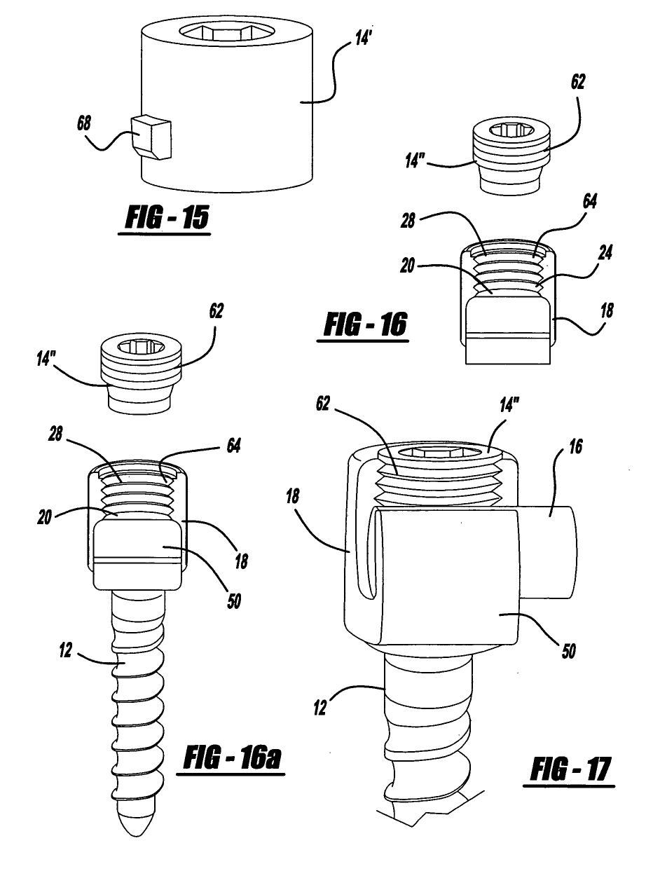

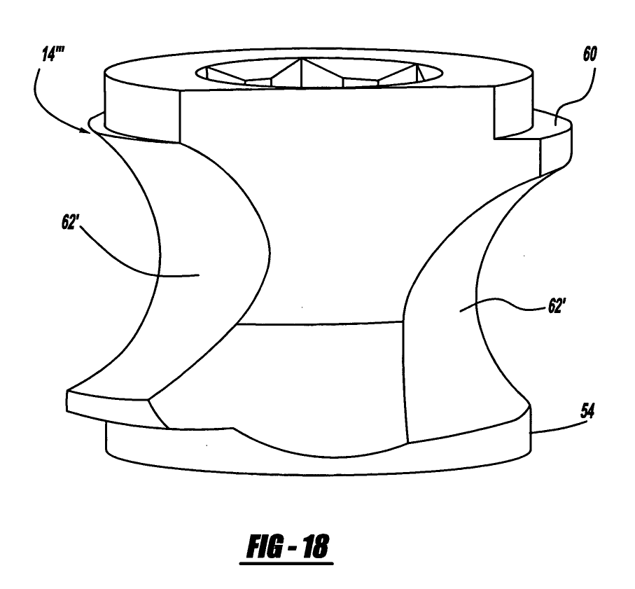

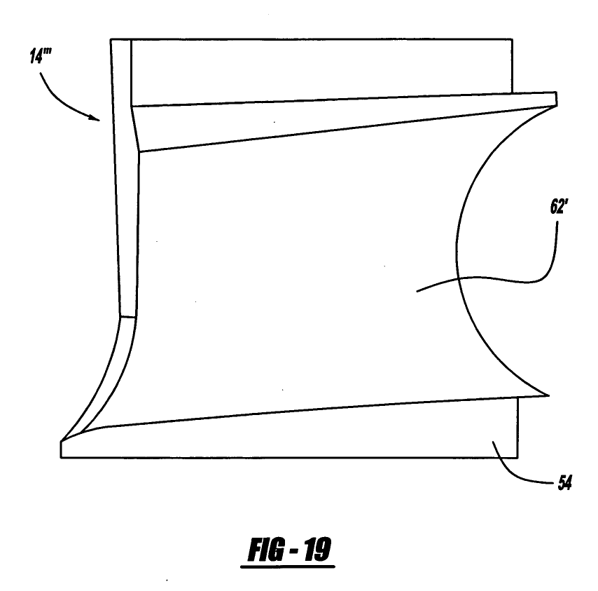

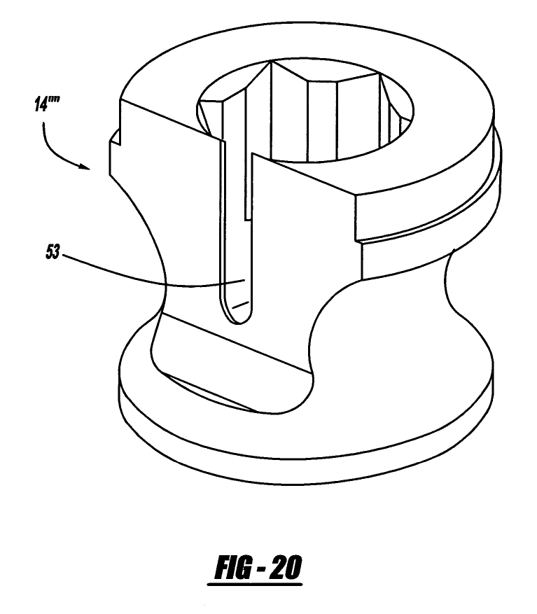

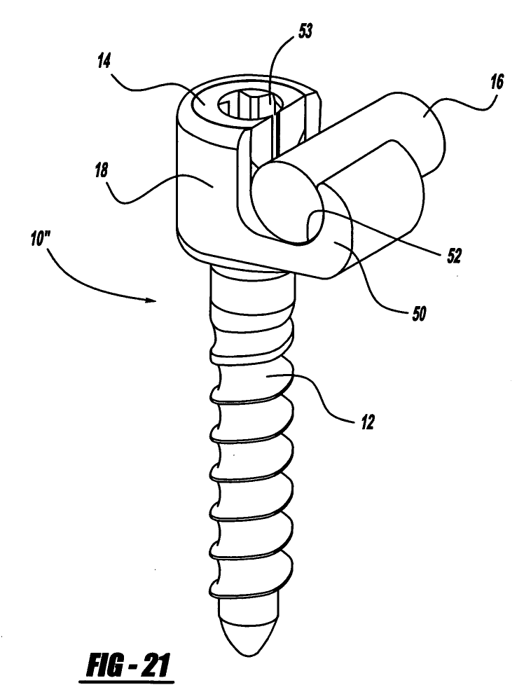

The locking mechanism 14 can be a cam lock, or other similar locking mechanism as is known to those skilled in the art. FIGS. 3, 6, 8, 9, 10, 12, and 14-17 illustrate embodiments of the locking mechanism 14.

Specifically, the locking mechanism 14 is a substantially tubular body 54. The locking mechanism 14 includes an outer surface 56 that engages a rod 16 and rod seat 50. The locking mechanism works in conjunction with the body portion 18 wherein the body portion 18 accommodates the locking mechanism 14 therein. More specifically, the locking mechanism 14 includes one relieved side 56 thereby allowing the rod 16 clearance to enter the assembly 10 including a grooved portion that engages the rod when the locking mechanism 14 is rotated into position. The head 66 of the locking mechanism 14 is capable of being turned for instance with the head of a screw. The locking mechanism 14 moves freely in rotation until the rod 16 is inserted into the assembly 10. Locking occurs by rotating the locking mechanism 14 through a rotational angle. The amount of rotation can be controlled by stops and any rotational angle can be used depending upon the amount of interference used in the locking means, for example at 90 degrees.

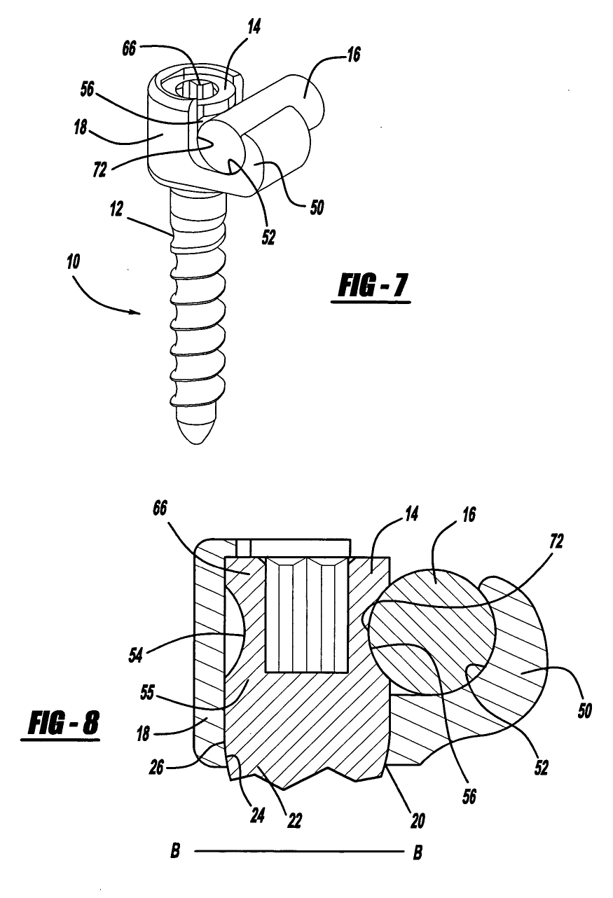

The locking mechanism 14 functions to secure and tighten the entire screw and rod fixation assembly 10. The locking mechanism 14 includes an outer surface 56 for engaging the rod 16. The outer surface 56 is formed to engage and maintain the rod 16 in the rod seat 50 within the body portion 18 as shown in FIGS. 3-8.

The locking mechanism 14 engages the rod 16 at the lower portion of the locking mechanism 14 as shown in FIG. 8. In other words, the locking mechanism 14 engages the rod 16 such that the rod 16 contacts the outer surface 56 of the locking mechanism 14 and forces the locking mechanism 14 against the screw head 22 while simultaneously forcing the rod 16 into the seat 50 because of the engagement. Preferably, the rod seat 50 is undersized relative to the rod diameter. The body portion 18 can also include a lip portion 74. The lip portion 74 can take many configurations, such as beads, a ring, or the like. However, this is not to be taken as limiting as other embodiments are possible. The lip 74, on a portion of the body portion 18, can be present to distribute the force more evenly over the screw head 22. Interference with the rod forces the locking mechanism to cant at an angle, a lip 74 minimizes this. Using a rod seat 50 with interference, any upward distraction force on the rod 16 is contained by the rod seat 50. Therefore, the force is directed away from the center of the locking mechanism 14.

The use of the cam lock 14′ enables the rod 16 to be partially locked within the assembly 10 at a lower load to allow a surgeon to hold the screw 12 on the assembly 10 while allowing the assembly 10 to be moved up and down the rod 16 for compression or distraction. When the locking mechanism 14 has a wedge shaped outer surface 58 there can be included a locking tab 68 that engages a hole/slot 76 in the body portion 18 when the wedge 58 reaches a locking position.

An alternative embodiment of the locking mechanism 14′ is shown in FIGS. 9-14. In this form of the locking mechanism 14′ the outer surface 56 is formed as a wedge 58. The wedge shape enables the locking mechanism 14′ to engage an outer surface 72 of the rod 16. The extending arm 60 of the locking mechanism 14′ maintains the rod 16 in proper engagement with the rod seat 50. An extended hook-like arm 60 can be included on the locking mechanism. The arm 60 allows a spring-like deflection, the spring force being dependent upon wall thickness, material properties, and geometry.

Additional embodiments of the locking mechanism 14″ are shown in FIGS. 16 and 17. In these embodiments of the locking mechanism 14″, the outer surface 56′ includes threads 62. The threads 62 engage a lock gripping mechanism 64 of the body portion 18. The outer surface 56 can also be formed as a curved groove 62′. The groove 62′ can be helically shaped, resembling a thread winding around another thread. Such a configuration enables the locking mechanism 14′″ to gradually tighten the grip on the rod 16, thus creating a more specific engagement between the locking mechanism 14′″ and the rod 16.

Alternatively, the locking mechanism 14″″ can include a slot 53. The slot 3 allows the head 66 of the locking mechanism 14″″ to spring inward. The benefit of the configuration is the ease with which the locking mechanism 14″″ can be inserted into the body portion 18.

The body portion 18 includes an aperture or passageway 20 extending therethrough. Within the passageway 20 are gripping devices 24. The gripping devices 24 are preferably threads 28. A screw seat 21 is located at the end of the passageway 20 there is a screw seat 21. The passageway 20 and screw seat 21 defines an A-A axis. Perpendicular to and non-intersecting with the A-A axis is a rod receiving seat 50. The rod receiving seat 50 includes an inner channel 52 that engages the rod 16 and defines a second axis B-B. Axis B-B is perpendicular to axis A-A therewith. Hence, the rod seat 50 is “offset” from the screw head seat 21 in that the axes defined by each are non-intersecting. The rod seat 50 is curved to better receive the rod 16. The curvature of the rod sear 50 is preferably of a similar curvature as that of the rod 16. The angle of the rod 16 relative to the screw 12 need not be absolutely perpendicular, depending upon its use, as described below. The rod receiving seat 50 holds the rod 16 in a position parallel to the A-A axis.

The advantage of an offset rod seat 50 is that the height of the assembly 10 can be decreased. Since the rod 16 is not seated directly above the screw head 22, a considerable amount of rod height is subtracted from the assembly height. Additionally, by incorporating an offset rod seat 50 into the assembly 10, arms, as are used in the assemblies of the prior art, for maintaining the rod 16 in place are no longer required. The primary benefit from the removal of the arms is eliminating the spread of arms, which is a common problem for polyaxial screw body designs.

The offset rod seat 50 is preferably undersized relative to the diameter of the rod 16. The undersized rod seat 50 forces the rod 16 to engage the rod seat 50. The engagement with the rod seat 50 in conjunction with the engagement of the locking mechanism 14 enables the rod 16 to be held in proper alignment within the rod seat 50.

The rod 16 is maintained in proper alignment within the rod receiving seat 50 and the body portion 18 because it is held in place via a locking mechanism 14 including a head 66 and base 67 portion. The locking mechanism 14 is shaped to fit within the passageway 20 of the body portion 18. The head 66 of the locking mechanism 14 includes a relieved portion 57, which enables a rod 16 to be inserted into the assembly 10 after the locking mechanism 14 is placed in the assembly 10, and a grooved portion 56 that engages the rod 16. Also, the locking mechanism 14 includes an arm 60 that prevents the rod 16 from backing out of the rod receiving seat 50. The base 67 of the locking mechanism 14 includes a seat 55 for engaging the top of the screw head 22.

The grooved portion 56 of the locking mechanism 14 is only present on a portion of the head 66 of the locking mechanism 14, such that the locking mechanism 14 must be turned within the body portion 18 to engage the rod 16. The configuration of the locking mechanism 14 enables the assembly 10 to be fully assembled prior to insertion into a patient. The ability to fully assemble the assembly 10 outside of the patient enables the assembly 10 to be inserted into the patient as a single unit, thereby lessening the amount of maneuvering required to position the assembly 10 properly once the assembly 10 is inserted into the patient. Once the assembly 10 is in place, the locking mechanism 14 is twisted such that the grooved portion 56 of the locking mechanism 14 can engage the rod 16 and thus prevent movement of the rod 16.

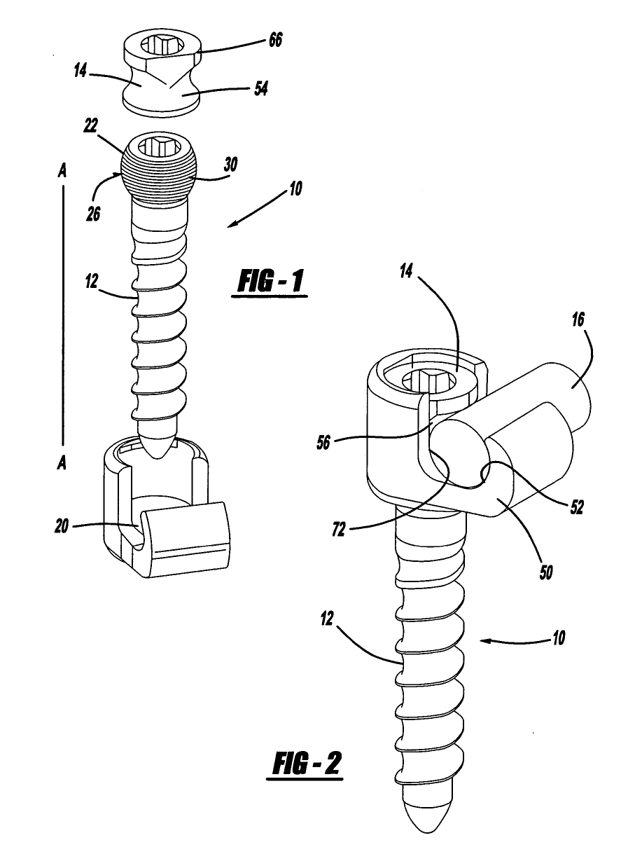

The body portion 18 includes an aperture/passageway 20 having a gripping portion 24 for threadedly gripping a boney surface. The body portion 18 operatively engages the screw head 22 and ultimately the screw 12. The body portion 18 is basically a passageway or single-walled vessel made of semi-flexible material. As described above, the body portion 18, and more particularly the gripping portion 24, securely grips the screw head 22 because the gripping portion 24 of the aperture includes threads 28. Additionally, the screw 12 includes threads 30. The threads 28, 30 of the body portion 18 and screw 12 grip each other and thereby prevent splaying of the threads when the threads are threadedly entrained together, as shown in FIG. 1. The “gripping” between the threads 28, 30 of the body portion 18 and screw 12 eliminate or at least significantly reduce movement of the screw 12 within the body portion 18.

The term “gripping” means that the threads not only engage each other in a direction parallel to the longitudinal axis 32 of the screw 12, but also engage each other in a direction 29 radially extending from the longitudinal axis 32. Thus, the screw 12 is longitudinally fixed and the gripping of the threads prevents separation of the threads in a radial direction 34, relative to the longitudinal axis 32.

The body portion 18 can have various shapes and designs. Although a substantially tubular body portion 18 is utilized with the present invention, any other similar body shapes known to those of skill in the art to function in the manner disclosed herein can be utilized with the present invention. The body portion 18 also varies in overall dimensions depending upon the desired use thereof. Further, the body portion 18 can be segmented so portions of the body portion 18 can be snapped off. Further, the body portion 18 can be threaded or smooth. The threading can be on the exterior surface or on the interior surface thereof. The threading is useful when the body portion 18 is used in conjunction with a similarly threaded locking mechanism 14 as is described in more detail below.

The screw 12 includes a body portion 13 having a threaded outer surface 26 as shown in FIG. 1. The threaded outer surface 26 of the screw 12 is in threaded engagement with the threaded aperture 20 so as to be screwed into locked engagement with the body portion 18. The threaded surface 26 of the screw 12 includes threads 30, which grip in mating engagement the threads 28 of the body portion 18. The “gripping” maintains the engagement of the screw 12 with the body portion 18.

More specifically, the threads 28, 30 of the present invention can be formed into any shape capable of creating a mating engagement. Alternatively shaped threads are well known to those of skill in the art.

In operation, the assembly 10 of the present invention is fully assembled prior to insertion into the body of the patient. In order to assemble the assembly 10, the screw 12 is placed within the passageway 20 of the body portion 18. The screw can either be screwed/locked into the screw seat 21 of the body portion 18 or the screw can remain unengaged until the locking mechanism 14 is placed within the passageway 20 of the body portion 18. When the screw 12 remains unengaged, the locking mechanism 14 is placed over the screw 12 within the passageway 20 of the body portion 18. The locking mechanism 14 and screw 12 can then simultaneously be engaged within the gripping devices 24 located in the passageway 20 of the body portion 18.

The locking mechanism 14 is engaged within the passageway 20 of the body portion 18 such that the relieved portion 57 of the locking mechanism 14 is facing the rod receiving seat 50. The rod 16 can then be freely inserted into the rod receiving seat 50 of the assembly 10. Once the rod 16 is inserted into the rod 16 receiving seat 50, the locking mechanism 14 is turned such that the grooved portion 56 of the locking mechanism 14 is brought into engagement with the outer surface 72 of the rod 16 within the rod receiving seat 50. When the rod 16 is fully engaged by the locking mechanism 14, the assembly 10 can be inserted into the body of the patient. Alternatively,

More specifically, the present invention provides a device 10 for locking a screw 12 within an aperture 20 of a body portion 18 without requiring the body portion 18 to be altered. In other words, the body portion 18 does not have to be physically manipulated, such as by the addition of a nut to the exterior surface of the body portion 18. The body portion 18 grips the screw head 22 rigidly, preventing movement of the screw head 22 that can occur because of vertebrae movement, without application of force or manipulation of the body portion 18 to ensure the fixed gripping.

The components for the screw fixation assembly and device disclosed and described herein can be manufactured by various methods known to those of skill in the art. For example, the assembly and device can be made by first blanking the outer shape from a round bar stock. Then, by holding on the threaded end or an extension of the threaded end, a hole is made in the opposite end. This hole is undersized relative to the taper to allow the taper to be cut with a single tool. While the part turns in a lathe, a boring bar having a small cutting tip is introduced into the hole and the taper and recess cut. The threads are then cut, any extension cut off, and the slot either milled or cut to be more compatible.

The various other components can be made by cutting the outside cylindrical shape with an extension to hold the components on a lathe. A hole is drilled into one end and a boring bar with a small cutting tip is used to enter the hole and cut the spherical seat. The outer slots are cut by slitting away the material with either a saw or a wire.

Throughout this application various publications are referenced by author and year. United States patents however, are referenced by number and inventor. Full citations for the publications are listed below. The disclosures of these publications and patents in their entireties are hereby incorporated by reference into this application to more fully describe the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

1. A screw and rod fixation assembly for fixing a screw and a rod comprising:
a screw including a screw head;
a body portion including screw head receiving means for receiving said screw head and rod receiving means for receiving a rod therein, said rod receiving means being offset from said screw head receiving means; and
locking means for locking a rod within said rod receiving means.

2. The screw and rod fixation assembly according to claim 1, wherein said locking means has an engaged condition for engaging and maintaining the rod in position in said rod receiving means and an unengaged condition for enabling the rod to freely enter said rod receiving means.

3. The screw and rod fixation assembly according to claim 2, wherein said locking means includes a relieved wall for positioning said locking means in said unengaged condition.

4. The screw and rod fixation assembly according to claim 2, wherein said locking means includes a grooved wall for positioning said locking means in said engaged condition.

5. The screw and rod fixation assembly according to claim 4, wherein said grooved wall includes rod engaging means.

6. The screw and rod fixation assembly according to claim 5, wherein said rod engaging means is selected from the group consisting essentially of threads, a helical groove, and a wedge shape.

7. The screw and rod fixation assembly according to claim 1, wherein said locking means is a cam lock.

8. The screw and rod fixation assembly according to claim 7, wherein said cam lock includes a wedge shaped outer surface.

9. The screw and rod fixation assembly according to claim 8, wherein said wedge shaped outer surface further includes a locking tab.

10. The screw and rod fixation assembly according to claim 8, wherein said cam lock further includes extraction means for extracting said cam lock from said body portion.

11. The screw and rod fixation assembly according to claim 7, wherein said cam lock includes gripping means on an exterior surface of said cam lock for maintaining said cam lock in said body portion.

12. The screw and rod fixation assembly according to claim 11, wherein said cam lock gripping means includes threads.

13. The screw and rod fixation assembly according to claim 11, wherein said locking means includes a screw head seat.

14. The screw and rod fixation assembly according to claim 13, wherein said screw head seat is formed in a shape selected from the group consisting essentially of conical and spherical.

15. A rod receiving device comprising a body portion including screw receiving means for receiving a screw therein and rod seating means offset from said screw receiving means within said body portion and locking means for locking a rod within said body portion.

16. The rod receiving device according to claim 15, wherein said locking means has an engaged condition for engaging and maintaining the rod in position in said rod receiving means and an unengaged condition for enabling the rod to freely enter said rod receiving means.

17. The rod receiving device according to claim 16, wherein said locking means includes a grooved wall for positioning said locking means in said engaged condition.

18. The rod receiving device according to claim 16, wherein said engaged condition includes a grooved wall.

19. The rod receiving device according to claim 18, wherein said grooved wall includes rod engaging means.

20. The rod receiving device according to claim 19, wherein said rod engaging means is selected from the group consisting essentially of threads, a helical groove, and a wedge shape.

21. The rod receiving device according to claim 1, wherein said locking means is a cam lock.

22. The rod receiving device according to claim 21, wherein said cam lock includes a wedge shaped outer surface.

23. The rod receiving device according to claim 22, wherein said wedge shaped outer surface further includes a locking tab.

24. The rod receiving device according to claim 22, wherein said cam lock further includes extraction means for extracting said cam lock from said body portion.

25. The rod receiving device according to claim 21, wherein said cam lock includes gripping means on an exterior surface of said cam lock for maintaining said cam lock in said body portion.

26. The rod receiving device according to claim 25, wherein said cam lock gripping means includes threads.

27. The rod receiving device according to claim 25, wherein said screw head receiving means is an aperture having gripping means.

28. The rod receiving device according to claim 27, wherein said aperture gripping means includes threads capable of mating said threads of said cam lock.

29. Rod receiving means for receiving and maintaining a rod therein, said rod receiving means comprising a body portion including screw head receiving means for receiving a screw head and a rod seat for receiving a rod therein, said rod seat being offset from the screw head receiving means.

30. The rod receiving means according to claim 29, wherein said body portion includes gripping means for gripping and maintaining the screw head within said body portion.

31. The rod receiving means according to claim 30, wherein said gripping means includes threads.

32. A medical device comprising:
a screw including a screw head;
a body portion including screw head receiving means for receiving said screw head and rod receiving means for receiving a rod therein, said rod receiving means being offset from said screw head receiving means; and
locking means for locking a rod within said body portion.

33. The medical device according to claim 32, wherein said locking means has an engaged condition for engaging and maintaining the rod in position in said rod receiving means and an unengaged condition for enabling the rod to freely enter said rod receiving means.

34. The medical device according to claim 33, wherein said locking means includes a relieved wall for positioning said locking means in said unengaged condition.

35. The medical device according to claim 33, wherein said locking means includes a grooved wall for positioning said locking means in said engaged condition.

36. The medical device according to claim 35, wherein said grooved wall includes rod engaging means.

37. The medical device according to claim 36, wherein said rod engaging means is selected from the group consisting essentially of threads, a helical groove, and a wedge shape.

38. The medical device according to claim 32, wherein said locking means is a cam lock.

39. The medical device according to claim 38, wherein said cam lock includes a wedge shaped outer surface.

40. The medical device according to claim 39, wherein said wedge shaped outer surface further includes a locking tab.

41. The medical device according to claim 38, wherein said cam lock further includes extraction means for extracting said cam lock from said body portion.

42. The medical device according to claim 38, wherein said cam lock includes gripping means on an exterior surface of said cam lock for maintaining said cam lock in said body portion.

43. The medical device according to claim 42, wherein said cam lock gripping means includes threads.

44. The medical device according to claim 32, wherein said locking means includes a screw head seat.

45. The medical device according to claim 44, wherein said screw head seat is formed in a shape selected from the group consisting essentially of conical and spherical.

46. An insert element comprising:
a body portion including screw head receiving means for receiving a screw head and rod receiving means for receiving a rod therein, said rod receiving means being offset from said screw head receiving means; and
locking means for locking a rod within said body portion.

47. The insert element according to claim 46, wherein said locking means has an engaged condition for engaging and maintaining the rod in position in said rod receiving means and an unengaged condition for enabling the rod to freely enter said rod receiving means.

48. The insert element according to claim 47, wherein said locking means includes a relieved wall for positioning said locking means in said unengaged condition.

49. The insert element according to claim 47, wherein said locking means includes a grooved wall for positioning said locking means in said engaged condition.

50. The insert element according to claim 49, wherein said grooved wall includes rod engaging means.

51. The insert element according to claim 50, wherein said rod engaging means is selected from the group consisting essentially of threads, a helical groove, and a wedge shape.

52. The insert element according to claim 46, wherein said locking means is a cam lock.

53. The insert element according to claim 52, wherein said cam lock includes a wedge shaped outer surface.

54. The insert element according to claim 53, wherein said wedge shaped outer surface further includes a locking tab.

55. The insert element according to claim 52, wherein said cam lock further includes extraction means for extracting said cam lock from said body portion.

56. The insert element according to claim 52, wherein said cam lock includes gripping means on an exterior surface of said cam lock for maintaining said cam lock in said body portion.

57. The insert element according to claim 56, wherein said cam lock gripping means includes threads.

58. The insert element according to claim 46, wherein said locking means includes a screw head seat.

59. The insert element according to claim 58, wherein said screw head seat is formed in a shape selected from the group consisting essentially of conical and spherical.

60. A locking mechanism for locking a rod within an assembly, said locking mechanism comprising;
a lock body including a relieved portion for allowing entry of a rod into an assembly and a grooved portion for engaging and maintaining the rod within the assembly.

61. The locking mechanism for locking a rod within an assembly 60, wherein said grooved wall includes rod engaging means.

62. The locking mechanism for locking a rod within an assembly 60, wherein said rod engaging means is selected from the group consisting essentially of threads, a helical groove, and a wedge shape.

63. A rod receiving device comprising a body portion including screw receiving means for receiving a screw therein and rod seating means offset from said screw receiving means within said body portion.

64. The rod receiving device according to claim 63, wherein said rod seating means is substantially curvate in shape.

65. The rod receiving device according to claim 63, wherein said rod seating means is smaller than the diameter of the rod being placed therein.

66. The rod receiving device according to claim 63, wherein said road seating means is located substantially perpendicular to an axis bisecting said screw receiving means.
